(19) United States
(12) Patent Application Publication
(10) Pub. No.: US 2026/0096955 A1
(43) Pub. Date: Apr. 9, 2026

(54) AEROSOL ANTIPERSPIRANT PRODUCT

(71) Applicant: THE PROCTER & GAMBLE COMPANY, Cincinnati, OH (US)

(72) Inventors: Kerry Andrew VETTER, Cincinnati, OH (US); Shawn David MCCONAUGHY, Cincinnati, OH (US); Wade Charles HENKE, Mason, OH (US); David Frederick SWAILE, Cincinnati, OH (US); Adrianna Nicole HYATT, Loveland, OH (US)

(21) Appl. No.: 19/349,101

(22) Filed: Oct. 3, 2025

Related U.S. Application Data
(60) Provisional application No. 63/702,962, filed on Oct. 3, 2024.
(60) Provisional application No. 63/875,781, filed on Sep. 4, 2025.
(60) Provisional application No. 63/875,727, filed on Sep. 4, 2025.

(51) Int. Cl. A61K 8/04; A61K 8/02; A61K 8/20; A61K 8/23; A61K 8/26; A61K 8/31; A61K 8/37; A61K 8/44; A61K 8/58; A61K 8/86; A61K 8/92; A61Q 15/00

(52) U.S. Cl. CPC A61K 8/046; A61K 8/0241; A61K 8/20; A61K 8/23; A61K 8/26; A61K 8/315; A61K 8/37; A61K 8/44; A61K 8/585; A61K 8/86; A61K 8/922; A61Q 15/00; A61K 2800/31; A61K 2800/412; A61K 2800/621; A61K 2800/87

(57) ABSTRACT

An aerosol antiperspirant composition that includes a liquid propellant and an antiperspirant concentrate. The concentrate includes a particulate active premix, a carrier, and a fragrance. The particulate active premix composition has a basic aluminum chloride salt of formula Al₂(OH)_{3.778}Cl_{2.222} to Al₂(OH)_{5.048}Cl_{0.952}, an amino acid, an alkaline earth metal salt, and greater than 1% of an alkali metal salt and/or magnesium chloride.

### FIELD OF THE INVENTION

The present disclosure relates to an aerosol antiperspirant product comprising an aerosol product container and an antiperspirant composition. More particularly, the antiperspirant composition includes a propellant and a basic aluminum chloride salt of formula Al₂(OH)_{3.778}Cl_{2.222} to Al₂(OH)_{5.048}Cl_{0.952}, an amino acid, greater than 1% of an alkali metal salt and/or magnesium chloride, and optionally an alkaline earth metal salt.

### BACKGROUND OF THE INVENTION

Aerosol antiperspirants are popular personal care products designed to provide effective perspiration and odor control for users. The active ingredients in these formulations are typically aluminum-based compounds, such as the basic aluminum chloride salts: aluminum chlorohydrate (ACH), aluminum sesquichlorohydrate (ASCH), and aluminum dichlorohydrate (ADCH). These compounds interact with the electrolytes in sweat, forming a gel-like plug that temporarily blocks sweat glands, thus significantly reducing perspiration.

While Al—Zr salts are known for their high efficacy in other forms, their use in aerosol formulations has been prohibited by regulatory bodies due to concerns regarding inhalation safety. Consequently, aerosol antiperspirants primarily rely on aluminum chlorides, including ACH, ASCH, and ADCH. These alternatives generally exhibit lower efficacy compared to Al—Zr compounds, and thus merit further investigation.

To make aluminum-based active ingredients more efficacious, various treatments have been explored to enhance their hygroscopicity including thermal activation and the incorporation of amino acids and alkaline earth metal salts. However, excessive hygroscopicity can lead to challenges: if these aluminum-based actives absorb too much moisture from the air, they can become sticky and difficult to handle. In extreme cases, the antiperspirant (AP) active may deliquesce, which can form an unmanageable gel or liquid, making it difficult to manufacture antiperspirant products.

Moreover, aerosol antiperspirant products must not only demonstrate efficacy but also dry quickly upon application to prevent discomfort and wetness. It can also be important that these products feel good on a user's skin, are not perceptible, and do not leave a sticky or greasy residue on the skin or clothing.

Therefore, there is a need for an aerosol antiperspirant product having an aluminum-based antiperspirant active with optimized hygroscopicity where the product dries quickly and feels good on a user's skin.

### SUMMARY OF THE INVENTION

An aerosol antiperspirant composition, comprising: (a) liquid propellant; (b) an antiperspirant concentrate comprising: (i) a particulate active premix composition comprising: (1) a basic aluminum chloride salt of formula Al₂(OH)_{3.778}Cl_{2.222} to Al₂(OH)_{5.048}Cl_{0.952}; (2) an amino acid; (3) an alkaline earth metal salt; (4) greater than 1% of an alkali metal salt and/or magnesium chloride; and wherein the particulate active premix composition comprises a plurality of particles; (ii) a carrier; and (iii) a fragrance.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 shows turbidity as a function of dilution for active premix solutions with different amino acids;

FIG. 2 is a partial cross-sectional view of an antiperspirant product;

FIG. 3 is a cross-sectional view of one embodiment of a valve assembly suitable for use with the antiperspirant product of FIG. 2;

FIG. 4 is a representative chromatogram for Peaks I-V;

FIG. 5A shows an image from an SEM microscope of Aluminum Chlorohydrate (ACH);

FIG. 5B shows an image from an SEM microscope of Active Premix Example 1B;

FIG. 5C shows the change in mass vs. time for Active Premix Examples 1A (0% NaCl), 1B (5% NaCl), and 1C (10% NaCl);

FIG. 6A shows an image from an SEM microscope of Active Premix Example 2A;

FIG. 6B shows an image from an SEM microscope of Active Premix Example 2B;

FIG. 6C shows the change in mass vs. time for Active Premix Examples 2A (0% NaCl), 2B (5% NaCl), and 2C (10% NaCl);

FIG. 7A shows an image from an SEM microscope of Active Premix Example 3A;

FIG. 7B shows an image from an SEM microscope of Active Premix Example 3B;

FIG. 7C shows the change in mass vs. time for Active Premix Examples 3A (0% NaCl), 2B (5% NaCl), and 2C (10% NaCl);

FIG. 8 an image from an SEM microscope of Active Premix Example 4;

FIG. 9 an image from an SEM microscope of Active Premix Example 5; and

FIG. 10 an image from an SEM microscope of Active Premix Example 6.

### DETAILED DESCRIPTION OF THE INVENTION

Aerosol antiperspirant products containing aluminum-based salts are widely used. In these spray formulations, the active antiperspirant ingredient is typically an aluminum-based compound, as aluminum zirconium (Al—Zr) salts, which are known for their superior efficacy in reducing sweat, are prohibited by regulatory agencies for use in aerosol products. As a result, the aluminum-based actives commonly employed, such as the basic aluminum chlorides, tend to exhibit lower efficacy in sweat reduction.

Optimizing the hygroscopicity of aluminum-based active ingredients can enhance the product's efficacy. However, it is important to ensure that the active ingredient is not overly hygroscopic, as excessive moisture absorption can lead to deliquescence, making it difficult to incorporate the antiperspirant active into a viable formulation.

The effectiveness of aluminum-based actives can be improved through various treatments. One such method involves heating the active solution prior to spray drying, which enhances the concentration of smaller, more effective polymers within the formulation. In both aluminum-based and Al—Zr complexes, these smaller polymers are commonly referred to as band 3 or peak 4 (U.S. Pat. Nos. 4,359,456 and 10,526,210 describe heating aluminum active solutions). The relative concentration of these polymers is often measured by the band III/II ratio or the Peak 4/3 ratio, with values exceeding 0.75 generally indicating a more potent active ingredient. This process, known as “activation,” results in formulations that are characterized as “activated,” “enhanced,” or “improved” antiperspirant actives.

Various companies have attempted to develop effective aluminum-based antiperspirant actives. Procter & Gamble® introduced an activated aluminum chlorohydrate solution in Old Spice® Ever Clear, a water-based stick with a calcium to aluminum atomic ratio of 0.06 and a glycine to calcium molar ratio of 5.2 for stability. However, consumer preference for the dry feel of anhydrous products limited its acceptance. Additionally, attempts to create a particulate active premix via spray drying aluminum chlorohydrate, calcium chloride, and glycine were unsuccessful, as the mixture lacked sufficient hygroscopic properties for effective dissolution in sweat. Unilever®'s Degree® Advanced Antiperspirant Dry Spray includes aluminum sesquichlorohydrate activated with calcium chloride and glycine, which offers better hygroscopicity for rapid dissolution. However, the increased acidity of these actives poses risks of corrosive damage to metal components within aerosol dispensers and can degrade fragrance components.

It was found that a particulate active premix containing basic aluminum chloride salts of formula Al₂(OH)_{3.778}Cl_{2.222} to Al₂(OH)_{5.048}Cl_{0.952} (Al:Cl atomic ratios between about 0.90 and about 2.10) and amino acids, an alkali metal salt, and optionally an alkaline earth metal salt can have enhanced hygroscopicity and antiperspirant efficacy. Additionally, the active premix can have higher pH values compared to other hygroscopic actives, which can improve compatibility with a wide array of fragrances, additives, anhydrous formulas, processing equipment, and containers.

Additionally, it can be important to optimize how the composition feels upon application while also reducing noticeable residue. It was found that if the active is added at the highest level that is allowed under current regulations, then the product may be highly efficacious for sweat control, however, it may not have consumer acceptable feel and leave behind noticeable residue. It was found that it can be desirable to add polar solvents like PPG-14 butyl ether, C12-15 alkyl benzoate, isopropyl myristate, and/or hydrogenated castor oil to improve skin feel and provide residue masking.

Table 1 and Table 2, below, include active premixes containing basic aluminum chloride salts of formula Al₂(OH)_{3.778}Cl_{2.222} to Al₂(OH)_{5.048}Cl_{0.952} (Al:Cl atomic ratios between about 0.90 and about 2.10) and amino acids, an alkali metal salt, and an alkaline earth metal salt. Active Premix Example 1 in Table 1 was made by adding aluminum chlorohydrate solution (% Al: 12.79, % Cl: 8.30 concentration), calcium chloride solution (31.39% concentration), sodium chloride, and glycine to a 5 L reactor equipped with a mechanical stirrer, reflux condenser, and temperature controller. The reactor was then brought to 95° C. and the solution was stirred for at least one and up to 24 hours. The reactor was then cooled to room temperature, and the solution was filtered through a 0.7 μm filter. The filtrate solution was collected, and spray dried to isolate a powder. Spray drying was performed with a Bowen Engineering, INC. (BE-994) Spray Dryer using the following conditions: Inlet temperature: 205° C., Outlet Temperature 130-140° C., air pressure: 60 PSI, and Collector H₂O: 6.5 in. The collected powder was then milled using a Hosokawa Alpine® jet mill (model #: 30369) with the following particle size distribution: D₅₀: 13.4 microns, D₉₀: 23.3 microns, and 0% greater than 88 microns to form the active premix.

TABLE 1
Active Premixes

| | Ex. 1 | Comp. Ex. 1 | Comp. Ex. 2 |
|---|---|---|---|
| | Active | Active | Active |
| | Premix | Premix | Premix |
| | (wt. %) | (wt. %) | (wt. %) |
| Aluminum | 20.39 | 24.22 | 17.2 |
| Chloride | 23.91 | 20.37 | 23.8 |
| USP anhydrous Active level | 65.9 | 80.63 | 59.38 |
| (USP Assay) | | | |
| Ca | 4.07 | | 2.97 |
| Glycine | 10.1 | | 16.65 |
| Al:Cl atomic ratio | 2.02 | 1.5 | 1.22 |
| % Na | 3.24 | | |
| % NaCl | 5.37 | | |
| Ca:Al atomic ratio | 0.13 | | |
| Gly:Ca molar ratio | 1.32 | | |
| Active pH (15% solution | 4.7 | 3.3 | 3.4 |
| in water) | | | |
| Peak 4/3 ratio | 2.9 | 1.07 | |

Active Premix Examples X and Y in Table 2 were also made, similarly to the method for Active Premix Example 1.

TABLE 2
Premix Examples

| | Ex. X | Ex. Y |
|---|---|---|
| % Al | 8.95-9.16 | 18.15-19.97 |
| % Ca | 1.68-2.37 | 3.62-5.01 |
| % Glycine | 3.94-5.55 | 7.95-11.59 |
| % Na | 0.75-1.06 | 1.67-2.32 |
| % Cl | 10.29-11.83 | 21.98-24.83 |
| Active pH | 4.43-4.79 | 4.37-4.79 |
| GPC Peak 4:3 Ratio | 2.49-3.92 | 2.17-3.44 |
| Gly:Ca Molar Ratio | 0.89-1.66 | 0.85-1.68 |
| Ca:Al Atomic Ratio | 0.13-0.18 | 0.12-0.18 |

### Clinical Study

A randomized, double-blind, 4-treatment round robin study with 36 female subjects was performed to determine whether Comparative Examples A-B and F and Example C, described in Table 3 and Table 4, below, had in vivo efficacy. Subjects underwent a 17-day washout with marketed bodywash, Ivory® Unscented, for all bathing and use of Old Spice® Fiji body spray as deodorant. Subjects were instructed to not use any antiperspirant products in their underarms during washout, and this was confirmed by swabbing the axilla for presence of aluminum at the baseline visit. The female subjects were required to shave underarms 3 days prior to the baseline visit.

Hot room sessions (40° C., 35% RH) were conducted at baseline and 72-hours after the fourth treatment. Hot room sessions included a 40-minute seated sweat-collection, followed by two, 20-minute walking sessions with sweat collection. Subjects who produced ≥250 mg and ≤1100 mg of sweat/axilla for the average of both 20-minute sweat collections, and who had a right/left axillary sweat ratio in the range of 0.6 to 1.67 at the baseline visit were randomized to treatment. A 4-day treatment phase followed, where products were applied once a day, on-site using a two second application dose, in the morning, followed by a 72-hour hot room visit after the fourth application.

The average of the two 20-minute sweat collections were analyzed using a mixed model Ancova (Baseline, Side and Treatment as fixed and Subject as random) after a base 10 logarithm transformation of the data. Descriptive statistics (N, least-squares mean, standard error and 95% two-sided confidence intervals) were calculated by treatment group and evaluated by two-sided hypothesis testing with type I error rate of 5%. The antiperspirant composition pH was determined according to the pH Method, described herein.

TABLE 3

| | Comp. | Comp. | | | |
|---|---|---|---|---|---|
| | Prod- | Prod- | Exam- | Exam- | Exam- |
| | uct A | uct B | ple C | ple D | ple E |
| | (wt %) | (wt. %) | (wt. %) | (wt. %) | (wt. %) |
| Propellant Blend: | 60.00 | 60.00 | 60.00 | 60.00 | 60.00 |
| 61.67% A-17 propellant | | | | | |
| (n-Butane@>95%/ | | | | | |
| Isobutane@<5%) | | | | | |
| □ 38.33% | | | | | |
| hydrofluorocarbon | | | | | |
| 152A | | | | | |
| Cyclopentasiloxane | Q.S. | Q.S. | Q.S. | Q.S. | Q.S. |
| Comparative Example 1 | 11.40 | | | | |
| Comparative Example 2 | | 11.40 | | | |
| Example 1 Active | | | 12.35 | 8.36 | 16.34 |
| Premix | | | | | |
| PPG-14 Butyl Ether | 3.80 | 3.80 | 3.80 | 3.80 | 3.80 |
| Dimethicone (and) | 0.38 | 0.38 | 0.38 | 0.38 | 0.38 |
| Dimethiconol | | | | | |
| Disteardimonium | 1.14 | 1.14 | 1.14 | 1.14 | 1.14 |
| Hectorite | | | | | |
| Mineral Oil Light | 0.19 | 0.19 | 0.19 | 0.19 | 0.19 |
| White | | | | | |
| Propylene Carbonate | 0.38 | 0.38 | 0.38 | 0.38 | 0.38 |
| Envicap Aerosol Starch | 0.76 | 0.76 | 0.76 | 0.76 | 0.76 |
| AN with Feminine 4 | | | | | |
| 2017C | | | | | |
| Anhydrous Active in | 23.1 | 18.5 | 18.5 | 12.5 | 24.5 |
| Concentrate | | | | | |
| Product Concentrate pH | 4 | 4.0 | 4.8 | 4.8 | 4.8 |

TABLE 4

| Comparative Product F | INCI |
|---|---|
| Degree ® Advanced | Active Ingredient: Aluminum |
| Antiperspirant | Sesquichlorohydrate |
| Dry Spray Deodorant Dry | (17.6%) |
| Spray Sexy Intrigue ® | |
| Lot code 01034AX02 | Inactive Ingredients: Butane, |
| Exp. January 2026 | Cyclopentasiloxane, Hydrofluorocarbon |
| | 152a, Isobutane, PPG-14 Butyl Ether, |
| | Glycine, Fragrance (Parfum), |
| | Disteardimonium Hectorite, Calcium |
| | Chloride, Propane, BAHT, Propylene |
| | Carbonate, Sodium Starch |
| | Octenylsuccinate, Maltodextrin, |
| | Hydrolyzed Corn Starch |
| | Octenylsuccinate, Silica. |
| Product Concentrate pH | 4.5 |

The results from the study are in Table 5, below. It was found that Ex. A (containing aluminum sesquichlorohydrate) and Ex. B (containing aluminum dichlorohydrate) did not perform as well Ex. C (containing the inventive active premix) or Ex. F (Degree® product containing aluminum sesquichlorohydrate).

As shown in Table 5, Ex. C was statistically better at reducing sweat, as compared to Comparative Examples A and B. Example C and Comparative Example F were shown to have similar performance. However, Comparative Example F has a lower pH than Example C, which may be required for the stability of the aluminum sesquichlorohydrate active. However, this low pH can degrade fragrance and aerosol containers including the sprayer components and therefore Example C may be preferred over Comparative Example F.

TABLE 5

| | | Reduction | vs. Ex. B | vs. Ex. C | vs. Ex. C |
|---|---|---|---|---|---|
| | Sweat | from | Two-sided | Two-sided | Two-sided |
| | (mg) | Baseline | P-value | P-value | P-value |
| Comp. Ex. A | 293 | −33.6% | 0.7760 | 0.0049 | 0.0299 |
| Comp. Ex. B | 299 | −32.1% | | 0.0029 | 0.0168 |
| Ex. C | 232 | −47.4% | | | 0.4585 |
| Comp. Ex. F | 246 | −44.3% | | | |

### Consumer Test

In a blind study, 31-32 panelists applied either Example D (lower active premix) or Example E (higher active premix) from Table 3 to both underarms. After application, they evaluated the scent by smelling themselves and assessed the product's look and feel. Panelists rated their experiences on a scale of Poor (0), Fair (25), Good (50), Very Good (75), and Excellent (100). These ratings were averaged to calculate the overall ratings, with separate averages for application experience and product feel, as shown in Table 6 and Table 7 below. Following this evaluation, the panelist used wet wipes to remove the product from their underarms and patted them dry. Next, the panelist applied the product they did not use in the first leg to both underarms and repeated the assessment process.

The results in Table 6, which includes data from female consumers, and Table 7, which includes data from male consumers, indicate that both groups preferred the aerosol product that contained a lower level of active premix. Although the higher level of active premix was expected to provide better wetness protection, the overall rating for the product with the lower dose was directionally higher. This product also offered superior overall application experience and product feel. Additionally, the lower active product was statistically better at not leaving residue or white products. Therefore, if the goal is to optimize both skin feel and performance, it may be advisable to formulate with less than 24.5% anhydrous active, such as cyclopentasiloxane and/or dimethicone.

TABLE 6

| | Ex. D | Ex. E |
|---|---|---|
| Overall Rating | 66 | 59 |
| Overall application experience | 65* | 54 |
| Overall Product Feel | 66 | 56 |
| Not leaving residue or white marks | 67* | 51 |

*statistically significant

TABLE 7

| | Ex. D | Ex. E |
|---|---|---|
| Overall Rating | 63 | 56 |
| Overall application experience | 64 | 58 |
| Overall Product Feel | 70 | 63 |
| Not leaving residue or white marks | 77* | 55 |

*statistically significant

### Aerosol Antiperspirant Products

The aerosol antiperspirant spray product can include an antiperspirant composition housed in the canister of an aerosol dispenser. The antiperspirant composition can include an antiperspirant concentrate and a propellant. The antiperspirant concentrate can include antiperspirant neuter and a fragrance.

### Antiperspirant Concentrate

The antiperspirant concentrate can be anhydrous. The antiperspirant concentrate can include a basic aluminum chloride antiperspirant active and a carrier, and optionally an emollient, a bulking or suspending agent, and a fragrance. The antiperspirant concentrate can have a pH of about 3.5 to about 5.2, from about 3.6 to about 5.0, from about 3.7 to about 4.9, from about 3.8 to about 4.8, from about 4 to about 5, from about 4.25 to about 4.75, or from about 4.5, as determined by the pH Test Method, described herein. The active premix can have a pH≥3.5, ≥3.6, ≥3.75, ≥4.0, or ≥4.25, as determined by the pH Test Method, described herein.

### Particulate Active Premix Containing a Basic Aluminum Chloride Antiperspirant Active

Basic aluminum chlorides have the general formula Al_{y}(OH)_{3y-z}Cl_{z}·nH₂O. Of particular interest are the basic aluminum chloride salts of formula Al₂(OH)_{3.778}Cl_{2.222} to Al₂(OH)_{5.048}Cl_{0.952}. This includes aluminum chlorohydrate (ACH), aluminum sesquichlorohydrate (ASCH), and/or aluminum dichlorohydrate (ADCH), which are detailed in Table 8 below. Other characteristics of ACH, ASCH, and ADCH can be found in the United States Pharmacopoeia and National Formulary (USP/NF) in its monograph (2024).

The concentrate can include less than or equal to 25% of the basic aluminum chloride AP active. The concentrate can include from about 10% to about 25%, from about 12.5% to about 24.5%, from about 12.5% to about 20%, from about 12.5% to about 17.5%, or from about 12.5% to about 15% of the basic aluminum chloride AP active.

TABLE 8
Basic Aluminum Chlorides

| | | Aluminum to | | |
|---|---|---|---|---|
| | | Chloride | | |
| | | Atomic Ratio | Chemical Formula | |
| Compound Name | Abbreviation | (Al:Cl) | Range | Example |
| Aluminum | ACH | 1.91-2.10 | Al₂(OH)_{5.048}Cl_{.952} to | Al₂(OH)₅Cl |
| Chlorohydrate | | | Al₂(OH)_{4.953}Cl_{1.047} | |
| Aluminum | ASCH | 1.26-1.90 | Al₂(OH)_{4.947}Cl_{1.053} to | Al₂(OH)_{4.5}Cl_{1.5} |
| Sesquichlorohydrate | | | Al₂(OH)_{4.413}Cl_{1.587} | |
| Aluminum | ADCH | 0.90-1.25 | Al₂(OH)_{4.400}Cl_{1.600} to | Al₂(OH)₄Cl₂ |
| Dichlorohydrate | | | Al₂(OH)_{3.778}Cl_{2.222} | |

It was found that a particulate active premix containing aluminum chlorohydrate of formula Al₂(OH)_{5.05}Cl_{0.95} to Al₂(OH)_{4.95}Cl_{1.05}, (Al:Cl atomic ratios between about 1.91 and about 2.1) and amino acids, alkaline earth metal salts and/or magnesium chloride, and optionally an alkali metal salt can have enhanced hygroscopicity and antiperspirant efficacy. Additionally, the active premix can have higher pH values compared to other hygroscopic actives including aluminum sesquichlorohydrate, which can improve compatibility with a wide array of fragrances, additives, anhydrous formulas, processing equipment, and containers.

Each particle in the active premix can include aluminum and the alkali metal salt and/or magnesium chloride. The alkali metal or magnesium can be interdispersed with the aluminum and/or it can be in a surface coating that can be visualized using energy-dispersive X-ray (EDX) spectroscopy coupled with a scanning electron microscope (SEM). For instance, the alkali metal salt can be sodium chloride and each particle can contain aluminum, sodium, and chloride. The sodium can be interdispersed with the aluminum and chloride and/or the sodium can be present in a surface coating that can be visualized using an SEM. Similarly, each particle can contain aluminum, magnesium, and chloride. The magnesium can be interdispersed with the aluminum and chloride or it can be present in a surface coating that can be visualized using an SEM.

The active premixes can have an amino acid to alkaline earth metal molar ratio of <6, ≤5.5, ≤5.25, ≤5, ≤4.9, <3, ≤2, ≤1.75, ≤1.5, or ≤1.4. The active premixes can have an amino acid to alkaline earth metal molar ratio of ≥0.25, ≥0.5, ≥0.75, ≥0.90, ≥1.0, ≥1.1, ≥1.25, or ≥1.3. Without wishing to be bound by theory, it is believed that lower ratios can be more effective in producing active premixes with improved hygroscopic properties. On the other hand, higher ratios are believed to be beneficial in achieving longer periods of solution stability before drying the active premix to a particle and it can be desirable to adjust the ratio based on manufacturing conditions.

The active premix can have a pH of about 3.5 to about 5.2, from about 3.6 to about 5.0, from about 3.7 to about 4.9, or from about 3.8 to about 4.8, as determined by the pH Test Method, described herein. The active premix can have a pH≥3.5, ≥3.6, ≥3.75, or ≥4.0.

The active premix can have a maximum change in mass over 48 hours of ≥45%, ≥47%, ≥50%, ≥55%, ≥60%, ≥52%, ≥54%, ≥60%, ≥70%, or ≥80% according to the Water Vapor Sorption Method, described herein. In order to maintain the stability and processability required for practical application in manufacturing antiperspirant formulations and shipping of antiperspirant formulations and active salts it can be advantageous for the active premix to have a change in mass over 48 hours of ≤150%, ≤140%, ≤130%, ≤125%, ≤115%, ≤110%, ≤106%, ≤100%, ≤90%, ≤80%, ≤70%, ≤65%, or ≤60% according to the Water Vapor Sorption Test Method, described herein.

The polymer size distribution of the ACH in the active premix can be defined by size exclusion chromatography method using Gel Permeation Chromatography (GPC), described herein in the GPC Test Method. The active premix can have a peak IV/III (Band III/II) ratio of exceeding 0.75, ≥1, ≥1.5, ≥2, ≥2.1, ≥2.4, ≥2.5, ≥3, ≥3.25, ≥3.4, ≥3.5, ≥3.75, ≥3.85, ≥3.90, ≥4.0, ≥4.2, ≥4.4, ≥4.6, ≥4.8, ≥5.0, ≥5.2, ≥5.4, ≥5.6, ≥5.8, ≥6.0, ≥6.25, ≥6.5, ≥6.75, ≥7.0, ≥7.25, ≥7.5, ≥7.75, or ≥7.9 according to the GPC Test Method. Active premixes having a peak IV/III ratio exceeding 0.75 can be referred to as “activated” and the process of heating to increase the peak IV/III ratio can be referred to as “activation.” The peak IV/III ratio can be dependent on several factors including the level and type of alkaline earth metal salt, amount and type of amino acid, and temperature and time of heating in solution prior to drying.

Alkaline earth metal salts can include an alkaline earth metal cation and an anion that is acceptable for application to the skin. Alkaline earth metals are elements in Group 2 of the periodic table and include beryllium, magnesium, calcium, and strontium. The anions associated with the alkaline earth metals salts can include chloride, sulfate, nitrate, or other halogen anions. More specifically, the salts can include calcium chloride, calcium nitrate, calcium sulfate, strontium chloride, strontium sulfate and strontium nitrate. Among these, calcium chloride may be preferred. The composition can include from about 0.5% to about 13%, from about 1% to about 10%, from about 1.5% to about 7%, or from about 1.75% to about 5% alkaline earth metal salts.

The amino acids can include natural amino acids, unnatural amino acids (e.g., non-canonical), and/or derivatives thereof (e.g., betaines such as trimethylglycine). The amino acids can include alanine, arginine, asparagine, aspartic acid, beta-alanine, cysteine, glutamic acid, glutamine, glycine, trimethylglycine, histidine, isoleucine, leucine, lysine, methionine, phenylalanine, proline, serine, threonine, tryptophan, tyrosine, valine, or mixtures thereof. Among these, glycine, trimethylglycine, alanine, leucine, or mixtures thereof may be preferred. Among these, glycine, alanine, and/or trimethylglycine may be preferred. The composition can include from about 1% to about 25%, from about 2% to about 20%, or from about 3% to about 18%, of the amino acid.

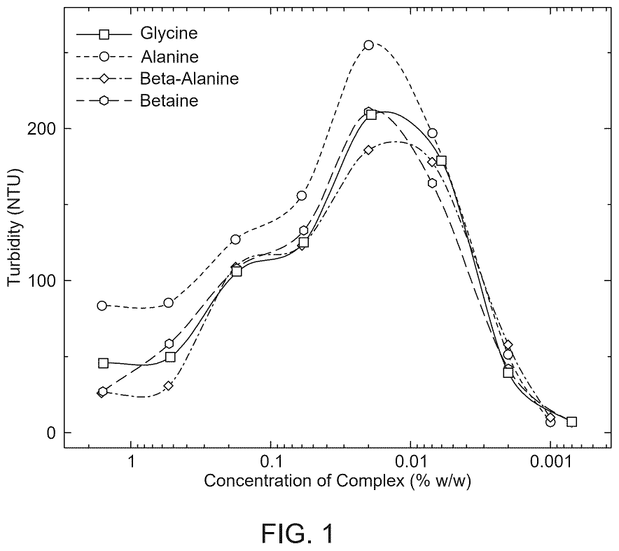

FIG. 1 shows the results of an experiment designed to evaluate the impact of various amino acids on an aluminum chlorohydrate active premix, which included calcium chloride, sodium chloride, and one of four amino acids: glycine, alanine, beta-alanine, or trimethylglycine.

In the experiment, 0.5 g of the active premix (APM) was combined with 10 g of synthetic sweat to create a stock solution. From this stock solution, eight sequential one-third dilutions were performed to prepare the test solutions. Specifically, Dilution 1 consisted of 5 g of the stock solution mixed with 10 g of synthetic sweat. Dilution 2 was made by combining 5 g of Dilution 1 with 10 g of synthetic sweat, and this process was repeated for a total of eight dilutions. Table 9 shows the dilutions.

After completing the dilutions, the test solutions were capped and allowed to equilibrate at ambient conditions for 16 hours. The turbidity of each test solution was then measured using a HACH 2100Q Portable Turbidimeter.

Table 9 and FIG. 1 show the results from this test and indicate that the turbidity serves as a proxy for precipitation in the sweat duct; greater turbidity indicates a more effective test solution. The results revealed that the active premixes containing glycine, alanine, beta-alanine, or trimethylglycine demonstrated similar performance characteristics when tested in vitro. It is believed that these amino acids, along with others, would be acceptable to consumers in the active premixes described herein. Additionally, some consumers may prefer naturally derived amino acids, including trimethylglycine.

TABLE 9

| Alanine | | Beta-Alanine | | Trimethylglycine | | Glycine | |
|---|---|---|---|---|---|---|---|
| APM | | APM | | APM | | APM | |
| Conc. | Turbidity | Conc. | Turbidity | Conc. | Turbidity | Conc. | Turbidity |
| (% w/w) | (NTU) | (% w/w) | (NTU) | (% w/w) | (NTU) | (% w/w) | (NTU) |
| 1.614 | 84 | 1.622 | 26 | 1.588 | 27 | 1.576 | 46 |
| 0.54 | 85 | 0.539 | 31 | 0.53 | 59 | 0.524 | 50 |
| 0.179 | 127 | 0.179 | 109 | 0.175 | 108 | 0.174 | 106 |
| 0.06 | 156 | 0.06 | 123 | 0.058 | 133 | 0.058 | 125 |
| 0.02 | 255 | 0.02 | 186 | 0.02 | 211 | 0.019 | 209 |
| 0.007 | 197 | 0.007 | 178 | 0.007 | 164 | 0.006 | 179 |
| 0.002 | 51 | 0.002 | 58 | 0.002 | 42 | 0.002 | 39 |
| 0.001 | 7 | 0.001 | 10 | 7.00E−04 | 7 | 7.00E−04 | 7 |

Moreover, the active premixes can have an alkaline earth metal to aluminum atomic ratio≥0.03, ≥0.05, ≥0.06, ≥0.09, or ≥0.10. The active premixes can have an alkaline earth metal to aluminum atomic ratio of ≤1, ≤0.75, ≤0.5, ≤0.4, ≤0.35, ≤0.25, or ≤0.2. The active premixes can have an alkaline earth metal to aluminum atomic ratio of about 0.03 to about 0.5, about 0.06 to about 0.35, or about 0.7 to about 0.25. Without wishing to be bound by theory, it is believed that higher ratios can create more activation or allow activation at lower temperatures. This higher ratio may also provide longer solution stability of the polymers before the active premix is dried to a particle. However, the higher ratio can generate higher absolute levels of glycine in the active premix that may, in turn, lead to a less hygroscopic active premix.

The active premixes can also include an alkali metal salt that is acceptable for use on skin. Alkali metals are elements in Group 1 of the periodic table and can include: lithium, sodium, potassium, or mixtures thereof. Anions for these salts of these metals can include chloride, sulfate, phosphates, nitrate, other halogen anions, or mixtures thereof. Acceptable salts can include sodium chloride, sodium sulfate, sodium phosphate, sodium nitrate, lithium chloride, lithium sulfate, lithium phosphate, lithium nitrate, potassium chloride, potassium nitrate, potassium sulfate, potassium phosphate, or mixtures thereof. The salts can include sodium chloride, potassium chloride, lithium chloride, or mixtures thereof. Among these, sodium chloride may be preferred. Without wishing to be bound by theory, it is believed that salts can further increase the water dissolution rate of the active premix. These salts can comprise up to 25% of the active premix, less than 15%, or less than 10%. If present, these salts can comprise at least 1% of the active premix, at least 2%, at least 3%, at least 4%, or at least 5%.

The active premixes can be made according to the following method: the basic aluminum chloride is present or dissolved in an aqueous solution that also contains the amino acid, and the alkaline earth metal, if present. Optionally, the solution can be heated to be activated, which is indicated by a peak IV/III ratio of exceeding 0.75. The time and temperature of heating will be dependent on the concentration of basic aluminum chloride in solutions during heating, the alkaline earth metal mole ratio, and the amino acid to alkaline earth metal molar ratio. Heating temperatures are 70° C. to 95° C. and heating time range from 1-24 hours. Heating temperatures, heating times, the alkaline earth metal atomic ratio, and the amino acid to alkaline earth metal molar ratio can be varied based on desired peak IV/III ratio, desired degree of hygroscopicity and available processing equipment. If present, the alkali metal salt can be added before, after, or independent of heating.

The particulate active premixes can be dried by any suitable manner including belt drying, tray drying and spray drying. Among these, spray drying may be preferred. The dried particle may be ground to desired particle size and/or sieved to desired particle distribution and may be spherical, platelet or random mixture in shape. The particle size may vary based on the final product (e.g., spray, cream, roll-on or stick product). The particulate active premix could be generally spherical in shape and can have a Dv50 average particle size of 25-35 microns. The particulate active premix could be generally platelet in shape and have a Dv50 average particle size of 12 to 18 microns. The active could be finely ground to have more than 85% or more than 90% (Dv90) of the particle having a size less than 10 microns and an average of less than 6 microns. Optionally, the active premix powder can also be dissolved or suspended in a solution, including aqueous, propylene glycol, propylene carbonate, or alcohol-based solution.

### Carrier

The carrier suitable can include an oil or a mixture of two or more oils. The carrier may be present in an amount from about 8% to about 35%, from about 10% to about 33%, from about 12% to about 30%, or from about 15% to about 24%, by weight of the composition.

Useful oils include, for example, volatile silicone oils and non-volatile organic oils. “Volatile silicone”, as used herein, refers to those silicone materials that have measurable vapor pressure under ambient conditions. Non-limiting examples of suitable volatile silicones are described in Todd et al., “Volatile Silicone Fluids for Cosmetics”, Cosmetics and Toiletries, 91:27-32 (1976). The volatile silicone can be a cyclic silicone having from at least about 3 silicone atoms or from at least about 5 silicone atoms but no more than about 7 silicone atoms or no more than about 6 silicone atoms. For example, volatile silicones can be used which conform to the formula:

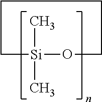

wherein n is from about 3 or from about 5 but no more than about 7 or no more than about 6. These volatile cyclic silicones generally have a viscosity of less than about 10 centistokes at 25° C. Suitable volatile silicones for use herein include, but are not limited to, Cyclomethicone D5 (commercially available from G. E. Silicones); Dow Corning 344, and Dow Corning 345 (commercially available from Dow Corning Corp.); and GE 7207, GE 7158 and Silicone Fluids SF-1202 and SF-1173 (available from General Electric Co.). SWS-03314, SWS-03400, F-222, F-223, F-250, F-251 (available from SWS Silicones Corp.); Volatile Silicones 7158, 7207, 7349 (available from Union Carbide); MASIL SF-V (available from Mazer) and combinations thereof. Suitable volatile silicone oils can also include linear silicone oils such as, for example, DC200 (1 cSt), DC200 (0.65 cSt), and DC2-1184, all of which are available from Dow Corning Corp. In certain examples, the volatile silicone oil can have a viscosity of less than 10 centistokes at 25° C.

### Emollient

The antiperspirant concentrate can also include an emollient. The concentrate can contain from about 2% to about 7.5%, about 2.25% to about 6.75%, about 2.5% to about 6%, about 2.75% to about 5.5%, or about 3% to about 5%, by weight of the composition. Too much emollient, in particular too much PPG-14, C12-15 alkyl benzoate, can leave the composition feeling greasy.

The organic emollient oils have a viscosity of less than 50 centistokes at 25° C. Non-limiting examples of nonvolatile organic emollients can include C12-15 alkylbenzoate (e.g., Finsolv® TN), butyl myristate, butyl stearate, cetyl octanoate, diethylhexylcyclohexane (e.g., Cetiol® S), dicaprylyl carbonate (e.g., Cetiol® CC), dicaprylyl ether (e.g., Cetiol® OE), hydrogenated castor oil, isobutyl stearate, isopropyl myristate, isostearyl isostearate, isostearyl lactate, isostearyl palmitate, mineral oil, myristyl myristate, neopentyl glycol diheptanoate (e.g., LexFeel® 7), octododecyl benzoate, octyldodecanol, petrolatum, PPG-14 butyl ether, or mixtures thereof.

The nonvolatile emollient could also comprise a silicone emollient such as dimethicone with a viscosity of 5 cst or greater. Dimethicone 5 cst or greater can be used in addition to a volatile silicone, like cyclopentasiloxane. Dimethicone 5 cst or greater can also replace the volatile silicone and in that case it can be present in the same concentrations as the carrier. Dimethicone 5 cst can be referred to as dimethicone (and) dimethiconol.

The emollient can be silicone free. The emollient can be miscible with volatile silicone.

### Bulking or Suspending Agents

The antiperspirant concentrate may contain one or more particulate bulking or suspending agents. The bulking or suspending agent may be hydrophobic, hydrophilic, or comprise mixtures thereof. In some specific embodiments, these materials may be hydrophilic in order to facilitate release of the antiperspirant active during use. Some examples of silica materials that may be used include, but are not limited to, colloidal silicas. Some non-limiting examples of silica materials are available from Evonik Industries under the trade names Aerosil 200SP, Aerosil 300SP, and Aerosil R972.

Some examples of clay materials that may be used at a low concentration include, but are not limited to, montmorillonite clays and hydrophobically treated montmorillonite clays. Montmorillonite clays are those which contain the mineral montmorillonite and may be characterized by a having a suspending lattice. Some examples of these clays include but are not limited to bentonites, hectorites, and colloidal magnesium aluminum silicates. Clay materials may be made hydrophobic by treatment with a cationic surfactant, such as a quaternary ammonium cationic surfactant. One example of a clay material is available from Elementis Specialities, Plc. of the UK under the trade name Bentone 38. A clay activator, such as propylene carbonate or triethyl citrate, may also be included in the antiperspirant

### Fragrance

An antiperspirant composition may optionally comprise fragrance comprising one or more perfume raw materials. The fragrance can be a free fragrance material and/or an encapsulated fragrance material. The antiperspirant composition can contain from about 0.6% to about 4.4%, from about 0.7% to about 4.0%, from about 0.8% to about 3.6%, from about 1% to about 3%, or from about 1.5% to about 2.5%, by weight of the antiperspirant composition, fragrance.

Free fragrance materials are typically liquids, which may contribute to the total amount of liquid materials in an antiperspirant composition. As used herein the term free fragrance material means a fragrance material that is not encapsulated, such as, for example, a mixture of perfume or aromatic components that are optionally mixed with a suitable solvent, diluent or carrier. Some suitable solvents, diluents or carriers for the perfume components may include ethanol, isopropanol, diethylene glycol monoethyl ether, dipropylene glycol, diethyl phthalate, triethyl citrate, and mixtures thereof. An antiperspirant composition may comprise from about 0.5%, 0.75% or 1% to about 4%, 3%, 2%, or 1.5% of a free fragrance material. An aerosol antiperspirant product may contain from about 0.5 g, 0.75 g, or 1 g to about 3 g, 2 g, or 1.5 g of free fragrance materials.

The fragrance may include one or more encapsulated fragrance materials for masking malodors, absorbing malodors, or provide the antiperspirant concentrate with a desired aroma during use. As used herein, the phrase “encapsulated fragrance material” refers to perfume components and the carrier encapsulating the perfume components. Encapsulated perfume materials also refer to carriers capable of absorbing a fragrance or malodor in use, such as for example an uncomplexed cyclodextrin material. The encapsulated perfume components may be released by a moisture activation mechanism whereby upon being wetted, e.g., by perspiration or other body fluids, the encapsulated perfume component is released. Alternatively or in addition thereto, the perfume components may be released by mechanochemical-fracture of the carrier, such as by the application of pressure, a shear force, or other event which releases the perfume component due to application of a force to the carrier. Encapsulated fragrance materials may be provided in a particulate form which would be considered part of the total particulate concentration of the antiperspirant composition.

An antiperspirant composition may comprise from about 0.25% to about 5%, or from about 0.5% to 5%, or from about 0.5% to about 4% by weight of the antiperspirant composition of an encapsulated fragrance material. Examples of some carriers suitable for forming the encapsulated fragrance materials include, but are not limited to, oligosaccharides (e.g., cyclodextrins), starches, polyethylenes, polayamides, polystyrenes, polyisoprenes, polycarbonates, polyacrylates, vinyl polymers, silicas, and aluminosilicates. Some examples of encapsulated fragrance materials are described in USPNs 2010/0104611; 2010/0104613; 2010/0104612; 2011/0269658; 2011/0269657; 2011/0268802; U.S. Pat. Nos. 5,861,144; 5,711,941; 8,147,808; and 5,861,144.

A perfume component may be any natural or synthetic perfume component known to one skilled in the art of creating fragrances including, but not limited to, essential oils, citrus oils, absolutes, resinoids, resins, concretes, etc., and synthetic perfume components such as hydrocarbons, alcohols, aldehydes, ketones, ethers, acids, esters, acetals, ketals, nitriles, etc., including saturated and unsaturated compounds, aliphatic, carbocyclic and heterocyclic compounds. Some non-limiting examples of perfume components include: geraniol, geranyl acetate, linalool, linalyl acetate, tetrahydrolinalool, citronellol, citronellyl acetate, dihydromyrcenol, dihydromyrcenyl acetate, tetrahydromyrcenol, terpineol, terpinyl acetate, nopol, nopyl acetate, 2-phenylethanol, 2-phenylethyl acetate, benzyl alcohol, benzyl acetate, benzyl salicylate, benzyl benzoate, styrallyl acetate, amyl salicylate, dimethylbenzyl carbinol, trichloromethylphenyl-carbinyl acetate, p-tert.butyl-cyclohexyl acetate, isononyl acetate, vetiveryl acetate, vetiverol, alpha-n-amylcinammic aidehyde, alpha-hexylcinammic aldehyde, 2-methyl-3-(p-tert.butylphenyl)-propanol, 2-methyl-3-(p-isopropylphenyl)-propanal, 3-(p-tert.butylphenyl)-propanal, tricyclodecenyl acetate, tricyclodecenyl propionate, 4-(4-hydroxy-4-methylpentyl)-3-cyclohexene carbaldehyde, 4-(4-methyl-3-pentenyl)-3-cyclohexene carbaldehyde, 4-acctoxy-3-pentyltetrahydropyran, methyldihydrojasmonate, 2-n-heptylcyclopentanone, 3-methyl-2-pentylcyclopentanone, n-decanal, 9-decenol-1, phenoxyethyl isobutyrate, phenyl-acetaldehyde dimethyl acetal, phenylacetaldehyde diethyl acetal, geranonitrile, citronellonitrile, cedryl acetate, 3-isocamphylcyclohexanol, cedryl methyl ether, isolongifolanone, aubepine nitrile, aubepinc, heliotropine, coumarin, eugenol, vanillin, diphenyl oxide, hydroxycitronellal, ionones, methylionones, isomethylionones, irones, cis-3-hexenol and esters thereof, indane musk fragrances, tetralin musk fragrances, isochroman musk fragrances, macrocyclic ketones, macrolactone musk frangrances, ethylene brassylate, aromatic nitro-musk fragrances. Some perfume components are also described in Arctander, Perfume and Flavour Chemicals (Aroma Chemicals), Vol. I and II (1969) and Arctander, Perfume and Flavour Materials of Natural Origin (1960).

### Propellant

The propellant can be a liquid propellant. An aerosol antiperspirant product can include a liquid propellant stored in a reservoir of the product container. The liquid propellant may be stored in the same reservoir as an antiperspirant concentrate or a separate reservoir. The propellant may be provided as a pressurized, liquefied gas which is at least partially miscible in a non-volatile silicone fluid of the antiperspirant concentrate. The propellant is utilized to drive the antiperspirant concentrate out of the product container during use and to assist with atomizing the antiperspirant concentrate as it exits the product container via the vaporization and expansion of the liquid propellant.

The antiperspirant product can contain from about 60% to about 90% or about 60% to about 80% by weight of the composition (i.e., propellant, antiperspirant neuter, and fragrance) of the liquid propellant.

The liquid propellants can have a boiling point (at atmospheric pressure) within the range of from about −45° C. to about 5° C. The propellants can be liquefied when packaged in the container under pressure. The rapid expansion of the propellant upon leaving the product container aids in the atomization of the antiperspirant composition. Suitable propellants may include chemically-inert hydrocarbons such as propane, n-butane, isobutane and cyclopropane, and mixtures thereof, as well as halogenated hydrocarbons such as dichlorodifluoromethane (propellant 12) 1,1-dichloro-1,1,2,2-tetrafluoroethane (propellant 114), 1-chloro-1,1-difluoro-2,2-trifluoroethane (propellant 115), 1-chloro-1,1-difluoroethylene (propellant 142B), 1,1-difluoroethane (propellant 152A), dimethyl ether and monochlorodifluoromethane, and mixtures thereof. Some propellants suitable for use include, but are not limited to, A-46 (a mixture of isobutane and propane), A-31 (isobutane), A-17 (n-butane), A-108 (propane), AP70 (a mixture of propane, isobutane and n-butane), AP40 (a mixture of propane, isobutene and n-butane), AP30 (a mixture of propane, isobutane and n-butane), and 152A (1,1 difluoroethane). Some aerosol antiperspirant products may incorporate an A-17 propellant and 152A. Other aerosol antiperspirant products may incorporate butane, isobutane, and propane.

The propellant may provide a pressure within a product container from about 60 kPa to about 500 kPA, 200 kPa to about 400 kPa, or 200 kPa to about 350 kPa at 25° C. The propellant may provide a pressure within a product container from about 100 kPa to about 1100 kPA, 200 kPa to about 1,100 kPa, or 400 kPa to about 900 kPa at 55° C.

### Product Container

The aerosol antiperspirant product container refers to the complete aerosol package intended to store and dispense an antiperspirant neuter and propellant. An aerosol antiperspirant product container may typically include at least one reservoir for storing the antiperspirant composition, a valve for controlling flow of the antiperspirant concentrate and liquid propellant, and an actuator by which a user can actuate the valve.

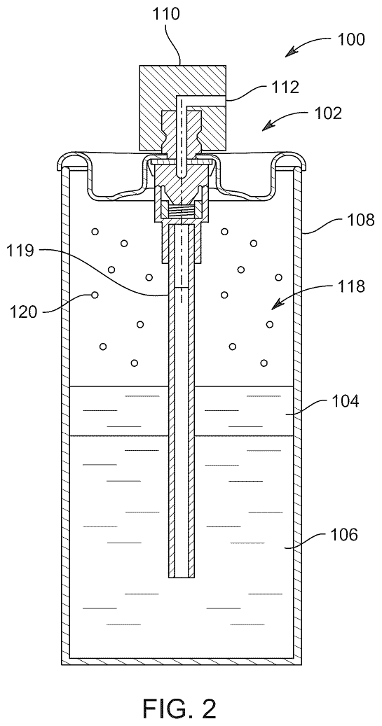

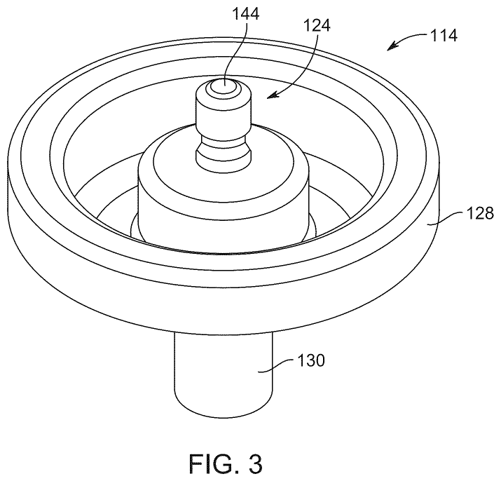

The aerosol antiperspirant composition described herein may be incorporated into a container or package. Referring to FIGS. 2-3, one example of an aerosol antiperspirant product is shown. The aerosol antiperspirant product 100 comprises a container 102, a liquid propellant 104, and an aerosol antiperspirant neuter 106. The container 100 comprises a body 108, an actuator 110 having a discharge orifice 112, a valve assembly 114, a dip tube 119, and a reservoir 118 that stores the liquid propellant 104 and the antiperspirant neuter 106. While one reservoir is shown, a plurality of reservoirs may be provided. The actuator 110, valve assembly 114, and container 100 may be provided in a wide variety of configurations, shapes, and sizes. The volume of the reservoir 118 may be from about 20 mL to about 120 mL, or from about 40 mL to about 110 mL, or from about 70 mL to about 110 mL. An aerosol antiperspirant product may contain from about 10 g to about 60 g, or from about 15 g to about 50 g, or from about 25 g to about 50 g of total materials stored in the reservoir 118.

When a user depresses the actuator 110, a valve within the valve assembly 114 is opened thereby reducing the pressure in the reservoir 118. As the pressure drops, the liquid propellant 104 begins to boil thereby maintaining/increasing the pressure in the reservoir 118 which forces some of the antiperspirant composition 106 and liquid propellant 104 up the dip tube 119, through the valve, and out of the discharge orifice 112 of the actuator 110. The liquid propellant mixed with the antiperspirant composition expands (within the actuator, upon exiting the actuator, or both) and atomizes the antiperspirant composition into droplets and forms a spray comprising the droplets and the gaseous propellant.

FIG. 3 shows a valve assembly 114 which may be attached to the body 108. The valve assembly 114 comprises a stem 124 to which the actuator 110 attaches, a mounting flange 128 for attaching the valve assembly 114 to the body 108, and a housing 130 attached to the mounting flange 128. The housing 130 contains a spring that biases the stem 124. The bottom portion of the housing 130 comprises a counter bore for receiving the dip tube 119. The axial bore 144 communicates with the actuator 110 when it is attached to the stem 124. When the actuator 110 is depressed, the scaling surfaces separate, thereby permitting a mixture of liquid propellant and antiperspirant neuter to flow through the radial bore to the axial bore 144 and onto the actuator 110.

The valve assembly 114 may comprise a vapor tap for diverting some of the gaseous propellant from the headspace of the reservoir 118 for the purpose of mixing the diverted gaseous propellant with the antiperspirant composition.

### Methods of Use

A user of an aerosol antiperspirant product may initiate a spray by depressing an actuator, thereby opening a valve in the product which enables a liquid propellant/antiperspirant composition mixture to exit the actuator. Prior to actuation, it may be desirable to shake or rotate the product to redisperse the liquid and particulate materials. While usage time can vary widely, users of an aerosol antiperspirant product may depress the actuator from about 2 seconds to about 5 seconds, or from about 2 seconds to about 4 seconds, or from about 2 seconds to about 3 seconds to provide a burst of antiperspirant composition for deposition to an underarm skin surface. An aerosol antiperspirant product may be sized to provide a life cycle from about 60 seconds to about 200 seconds, or from about 70 seconds to about 150 seconds, for from about 90 seconds to about 130 seconds. Aerosol antiperspirant product life cycles within these ranges may provide from about 15 to about 50 two second uses per product.

Wetness protection/product efficacy may increase as the amount of antiperspirant active delivered to skin increases, assuming the increased amount of active is available for activity (e.g., does not flake off). An aerosol antiperspirant product may deliver a total mass flow rate less than 0.5 g/sec or from about 0.1 g/sec to about 0.5 g/sec, or from about 0.2 g/sec to about 0.4 g/sec, or from about 0.25 g/sec to about 0.35 g/sec. An aerosol antiperspirant product may deliver an antiperspirant composition mass flow rate less than 0.3 g/sec or from about 0.1 g/sec to about 0.3 g/sec.

The product is generally a leave-on composition that can provide lasting odor protection and freshness and the composition may provide 72 hours or more of odor control after application. The product can provide wetness protection, which can refer to the product's ability to help reduce or prevent excessive sweating and the resulting wetness under the arms.

### Test Methods

### pH Test Method

The pH of particulate active premixes and/or the actives are determined by creating a 15% solution of the powder in deionized water and measuring with a calibrated glass electrode and meter such as a Mettler Toledo® SevenCompact pH S210 or equivalent.

For an antiperspirant product the pH can be determined as follows. First, if the product is an aerosol, the pH of product concentrate was determined by piercing a small hole in the product to allow the propellant to evaporate for at least 24 hours, then removing the valve assembly to allow removal of the product concentrate. 10 grams of the product or the product concentrate was then mixed with 20 grams of deionized water. After mixing vigorously to dissolve the active, the mixture was centrifuged at 10,000 rpm for 10 minutes to separate the aqueous layer from the insoluble portions of the product concentrate. The pH of the aqueous phase is then determined using the same equipment as the active or active premix samples and reported as the pH of the product or product concentrate.

### GPC Test Method

The GPC Test Method is performed on sample active premix to quantify relative concentration of aluminum polymer populations present. The method is preferably performed on active or active premix but can also be performed on finished antiperspirant product if active or active premix is not available. Peaks corresponding to five characteristic polymer populations are identified and integrated, and ratios of relative areas of peaks of interest are calculated and reported.

### Sample Preparation

If sample active or active premix is available, it is dissolved or dispersed in 0.01-M nitric acid such that active is present at 1.0% w/w. If only sample finished antiperspirant product is available, it is dissolved or dispersed in 0.01-M nitric acid such that the active is present at 1.0% w/w. After dilution, the nitric acid solution may be filtered (for example, using a 0.45-μm nylon syringe filter) if necessary. In any case, only the resulting nitric-acid extraction solution is subsequently injected into the chromatograph.

### Apparatus and Procedure

GPC chromatographic separation is performed using 0.01-M nitric acid mobile phase, using a total 900 mm length of silica columns. Exemplary suitable apparatus consists of three consecutive μPorasil Columns, 3.9×300 mm, 10 mm packing (available from Waters, Milford, Massachusetts), plumbed in series. The chromatograph is equipped with a refractive-index detector. Exemplary suitable refractive-index detector is ERC RefractoMax 520 (available from IDEX, Oak Harbor, Washington). A 5-μL injection volume is used, and an exemplary suitable flow rate is 0.8 mL/min.

### Analysis

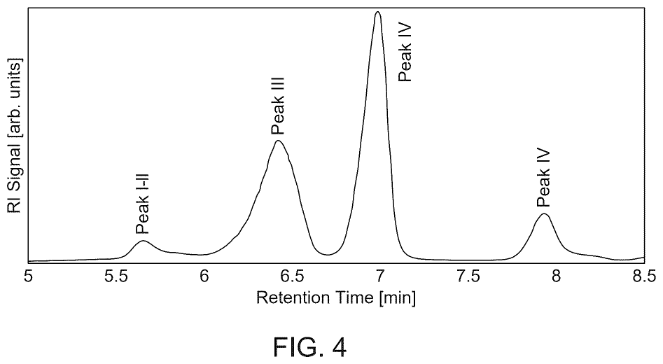

Chromatograms are processed and peaks integrated, for example using suitable software such as Thermo Fisher Scientific Chromeleon Data System. The focus of this analysis is a set of four or five peaks appearing early in the chromatogram that correspond to five characteristic polymer populations. (Sharper peaks, generally with longer retention time, are understood to correspond to small molecules present in the extracted active and are disregarded in this analysis.) The peaks observed in the chromatogram are designated in order of appearance on the chromatogram as Peaks I-II (generally appears as a single peak) and Peaks III, IV and V. A representative chromatogram containing the abovementioned peaks is shown in FIG. 4. The area of Peaks I-II, III, IV and V correspond to the relative concentration of aluminum polymer populations present in the analyzed active in the injected sample. (It is noted that these five polymer populations are generally recognized as present. Swaile U.S. Pat. No. 6,149,897 refers to the peaks in the chromatograms as Peaks I-II, Peak III, Peak IV, and Peak V. Similarly, Gosling U.S. Pat. No. 4,359,456 refers to Peak I-II as “Band 1,” Peak III as “Band 2, Peak IV as “Band 3”, and Peak V as “Band 4.”

Peaks I-II, III, IV, and V are each integrated, and their peak areas (arbitrary units) are recorded. The ratios between combinations of peaks of interest may be calculated and expressed as a dimensionless ratio. For example, the ratio of the area of Peak IV to the area of Peak III is calculated and reported as the “Peak IV/III ratio” to the nearest 0.01.

### Water Vapor Sorption Test Method

The Water Vapor Sorption Test Method is used to quantify the amount of water vapor uptake by a sample or composition when contained in a warm, moist environment. This method makes use of a dynamic vapor sorption (DVS) instrument to first dry and condition a specimen of a sample active premix or composition and then measure the increase of mass over time as the sample is held at elevated temperature and relative humidity.

A DVS instrument suitable for this method is capable of controlling percent relative humidity (% RH) to within +5% RH, temperature to within +2° C., and measuring mass to a precision of +0.01 mg. It is further capable of maintaining environments of 0% RH at both 37° C. and 50° C. and 90% RH at 37° C. One suitable exemplary apparatus is the S Endeavour Dynamic Vapor Sorption Analyzer (Surface Measurement Systems Ltd.), or equivalent. The DVS instrument is configured to record the mass of specimen at ten-minute intervals with a precision of 0.01 mg or better.

A 20.0±5.0 mg specimen of material is spread evenly on a tared aluminum sample pan appropriate for the DVS instrument. The specimen and pan are introduced into the DVS, and the specimen is first dried at 37° C. and 0% RH for 10 hours. The specimen is then conditioned at 37° C. and 0% RH for 20 hours. The specimen mass point recorded at 37° C. and 0% RH is taken as the initial specimen mass, denoted mᵢ. The specimen is then exposed to an environmental state at 37° C. and 90% RH for 18 hours. For any point in time/during the final 18 hours of the measurement, the percent change in mass of the specimen is calculated by the expression:

$$Percent change in mass [ \% ] = \frac{{m}_{t} - {m}_{i}}{{m}_{i}} \times 100 \%$$

where mₜ is the measured specimen mass at that point in time, and time/is the total time passed after the beginning of the first, drying condition of the DVS. The maximum percent change in mass of the specimen observed over the final 18 hours of the measurement, corresponding to time 30 hours to 48 hours of the DVS measurement procedure overall, is reported as the Change in Mass of the specimen, reported in percent to the nearest 1%.

### EXAMPLES

The examples in Table 10 and Table 11 were made as follows:

### ACH Active Premix (APM) Examples:

Aluminum chlorohydrate solution (40% anhydrous active), calcium chloride solution (32% w/w), and glycine were added to a glass reaction vessel equipped with a stir bar. The solution was allowed to stir until homogeneous. Following visible homogeneity, the reaction vessel was placed in a gravity oven and the solution was brought to 95° C. for at least one and up to 24 hours. The reaction vessel was then cooled to room temperature and the solution was isolated. The isolated solution was then divided into parts, and the appropriate amount of sodium chloride was then added to the premix solution to target 0%, 5%, and 10% in the powder product. Before spray drying, the solutions were filtered through a 0.7 μm filter. Following this, the resulting solutions were spray dried to isolate the corresponding powder. Spray drying was conducted using either a Büchi Mini Spray Dryer S-290 or a Büchi Mini Spray Dryer S-300 (Model: Corrosives 250° C. Pro) using the following conditions: Drying gas 30.5 m³/h, Spray gas 1200 L/h, Inlet temperature: 220° C., Outlet temperature 115-125° C., once spray drying was completed, the APM was collected in a vessel for further analysis using various analytical methods.

### ASCH Active Premix (APM) Examples:

Aluminum sesquichlorohydrate powder (82% anhydrous active), calcium chloride, and glycine were added to a 5 L reactor equipped with a mechanical stirrer, reflux condenser, and temperature controller. The reactor was brought to 95° C. for at least one and up to 24 hours. The reaction vessel was then cooled to room temperature, and the solution was isolated. The isolated solution was then divided into parts, and the appropriate amount of sodium chloride was then added to the premix solution to target 0%, 5%, and 10% in the powder product. Before spray drying, the solutions were filtered through a 0.7 μm filter. Following this, the resulting solution was spray dried to isolate the corresponding powder. Spray drying was conducted using either a Büchi Mini Spray Dryer S-290 or a Büchi Mini Spray Dryer S-300 (Model: Corrosives 250° C. Pro) using the following conditions: Drying gas 30.5 m³/h, Spray gas 1200 L/h, Inlet temperature: 220° C., Outlet temperature 115-125° C., once spray drying was completed, the APM was collected in a vessel for further analysis using various analytical methods.

### ADCH Active Premix (APM) Examples:

Ca/Gly activated ADCH solution was purchased commercially. The isolated solution was then divided into parts, and the appropriate amount of sodium chloride was then added to the premix solution to target 0%, 5%, and 10% in the powder product. Before spray drying, the solutions were filtered through a 0.7 μm filter. Following this, the resulting solution was spray dried to isolate the corresponding powder. Spray drying was conducted using either a Büchi Mini Spray Dryer S-290 or a Buchi Mini Spray Dryer S-300 (Model: Corrosives 250° C. Pro) using the following conditions: Drying gas 30.5 m³/h, Spray gas 1200 L/h, Inlet temperature: 220° C., Outlet temperature 115-125° C., once spray drying was completed, the APM was collected in a vessel for further analysis using various analytical methods.

The pH for the examples in Table 10 and Table 11 were determined according to the pH Test Method, described herein.

The Kinetic Study for the examples in Table 10 and Table 11 was performed according to the Water Vapor Sorption Test Method, described herein. The test determined the amount of water vapor sorption that occurs in a raw material.

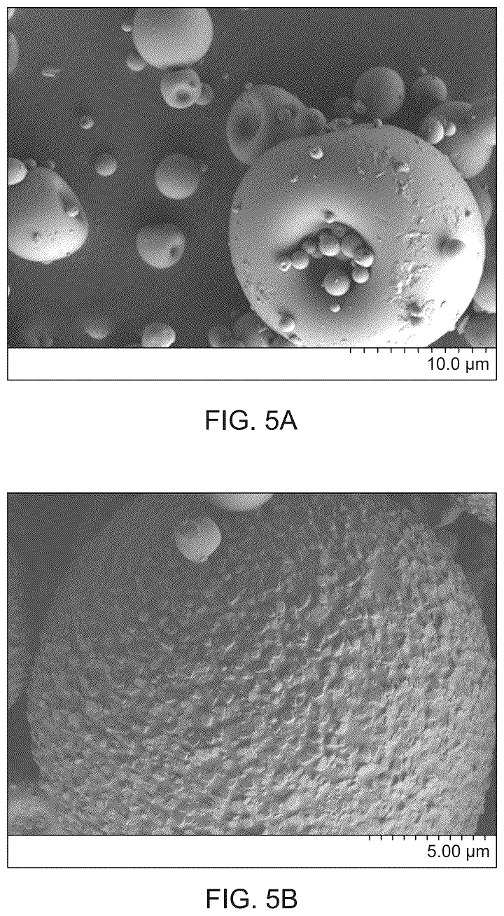

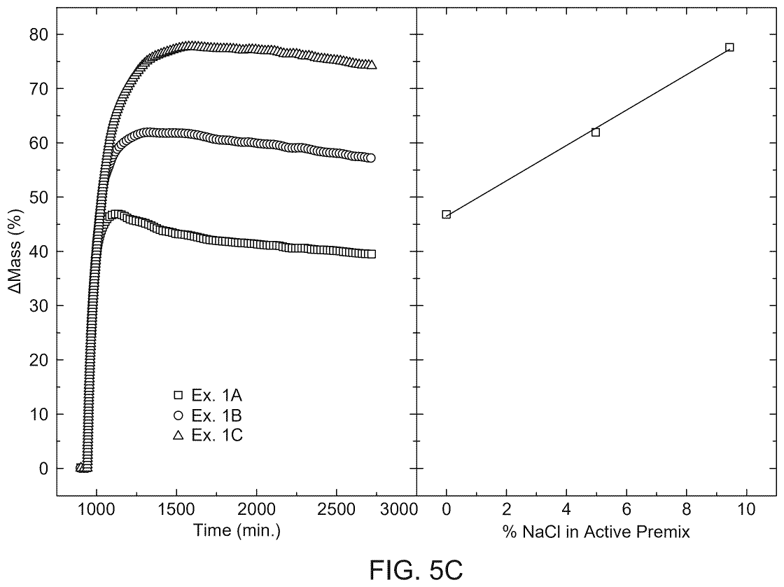

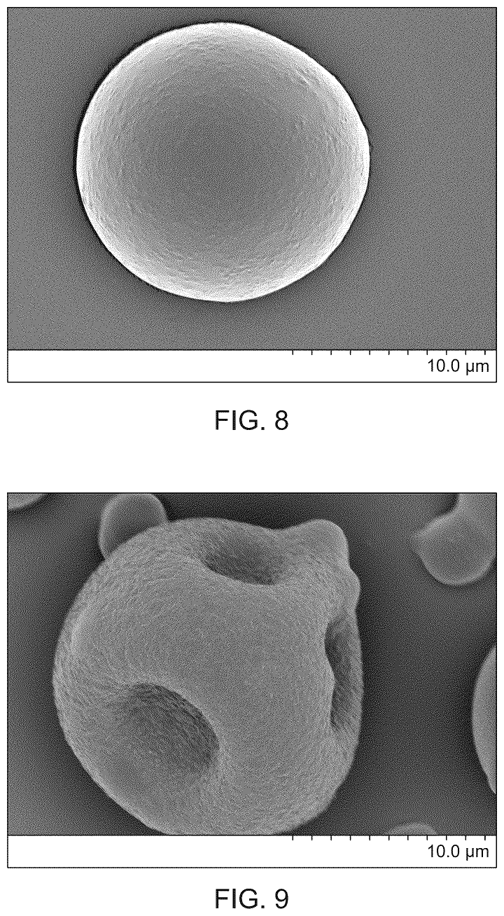

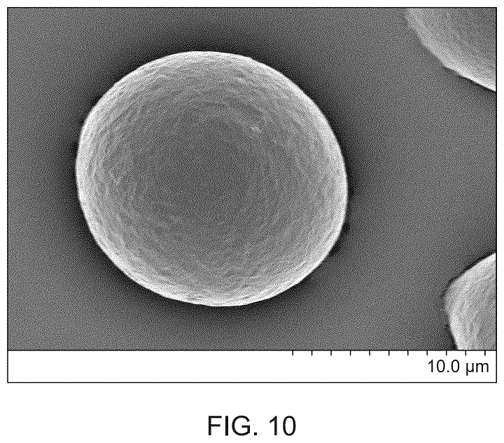

The SEM images shown in FIGS. 5A-5B, 6A-6B, 7A-7B, and 8-10 clearly show the effects of adding sodium chloride to the system. When no sodium chloride is added, the particles appear relatively smooth, as seen in FIGS. 5A, 6A, and 7A. However, after the addition of sodium chloride, the particles exhibit a rougher surface, with visible cubic sodium chloride crystals present, as shown in FIGS. 5B, 6B, 7B, and 8-10. Before spray coating, the particles in FIGS. 8-10 appeared similar to those depicted in FIG. 5A.

It was surprising to find sodium chloride deposits on the particle surfaces, especially since the spray-dried mixture is homogeneous and contains relatively low sodium compared to the amounts of aluminum (with a Na to Al atomic ratio of 0.12-0.28). In some instances, the sodium chloride covers a significant portion of the particle surface (e.g., more than a majority, >80% up to 95%) while in other cases, only about 50% may be covered, as shown in the SEM images.

FIGS. 5C, 6C, and 7C show that as the amount of sodium chloride added increases, the hygroscopicity also increases. These figures also surprisingly show that there is a linear dose response based on the amount of sodium chloride that is added. This indicates that sodium chloride enhances the moisture-absorbing properties of the active premix compared to formulations without sodium chloride, as demonstrated in APM Examples 1A, 2A, and 3A. The increased ability to absorb moisture from the environment due to the added sodium chloride is expected to facilitate a faster dissolution of the active premix in sweat, ultimately enhancing the effectiveness of the antiperspirant.

Additionally, a direct comparison of Examples 1B vs. 1C, 2B vs. 2C, and 3B vs. 3C illustrates that higher sodium chloride content correlates with greater hygroscopicity. However, it is important to recognize that while increased hygroscopicity can enhance the performance of the active premix, there is a trade-off. If the active premix becomes too hygroscopic, it can create challenges in shipping and incorporating it into antiperspirant formulations.

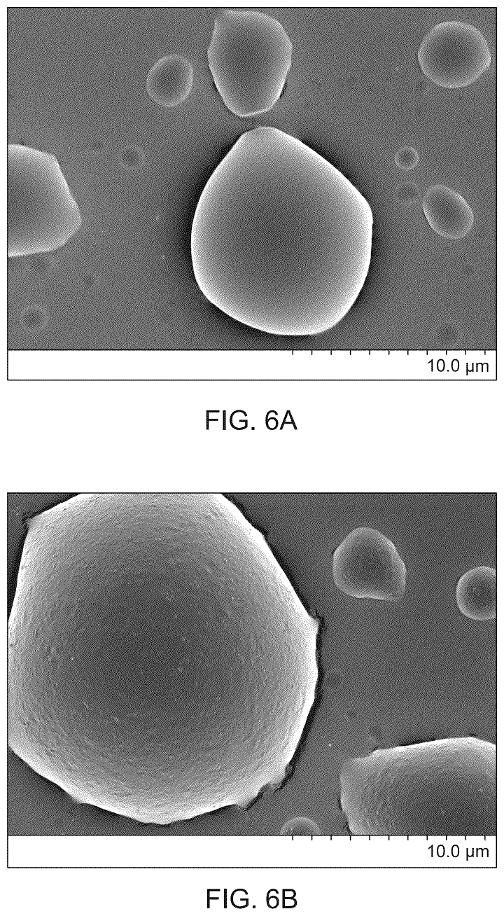

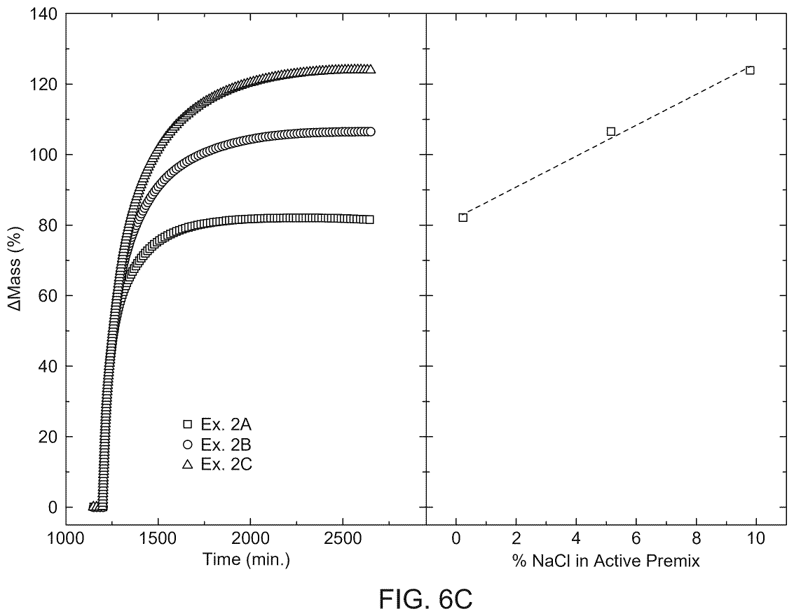

TABLE 10

| | Ex. | Ex. | Ex. | Ex. | Ex. | Ex. |
|---|---|---|---|---|---|---|
| | 1A | 1B | 1C | 2A | 2B | 2C |
| | APM | APM | APM | APM | APM | APM |
| Basic | ACH | ACH | ACH | ASCH | ASCH | ASCH |
| Aluminum | | | | | | |
| Chloride Salt | | | | | | |
| % Al | 21.3 | 19.2 | 18.1 | 17.8 | 17.2 | 16.2 |
| % Cl (Total) | 18.36 | 19.02 | 21.38 | 21.99 | 23.88 | 25.50 |
| % Ca | 2.17 | 1.99 | 1.87 | 3.9 | 3.82 | 3.6 |
| % Glycine | 12.3 | 17.7 | 17.2 | 17.90 | 15.59 | 14.15 |
| Target % | 20.52 | 20.05 | 19.53 | 19.00 | 18.60 | 18.19 |
| Glycine | | | | | | |
| Al:Cl Atomic | 1.52 | 1.33 | 1.11 | 1.06 | 0.95 | 0.83 |
| Ratio | | | | | | |
| Al:Cl Atomic | 1.93 | 2.02 | 1.93 | 1.56 | 1.62 | 1.61 |
| Ratio | | | | | | |
| of Active | | | | | | |
| % Na | 0 | 1.96 | 3.71 | 0.09 | 2.03 | 3.85 |
| Target % NaCl | 0 | 5 | 10 | 0 | 5 | 10 |
| Calculated % | 0.00 | 4.98 | 9.43 | 0.23 | 5.16 | 9.79 |
| NaCl | | | | | | |
| Ca:Al Atomic | 0.069 | 0.070 | 0.070 | 0.147 | 0.150 | 0.150 |
| Ratio | | | | | | |
| Gly:Ca Mol | 3.03 | 4.75 | 4.91 | 2.45 | 2.18 | 2.10 |
| Ratio | | | | | | |
| Active pH | | | | 4.19 | 4.33 | 4.39 |
| Peak 4:3 Ratio | | | | 6.49 | 6.63 | 6.69 |
| SEM Image | | FIG. | | FIG. | FIG. | |
| | | 5B | | 6A | 6B | |

| | | |
|---|---|---|
| Kinetic Study | FIG. 5C | FIG. 6C |

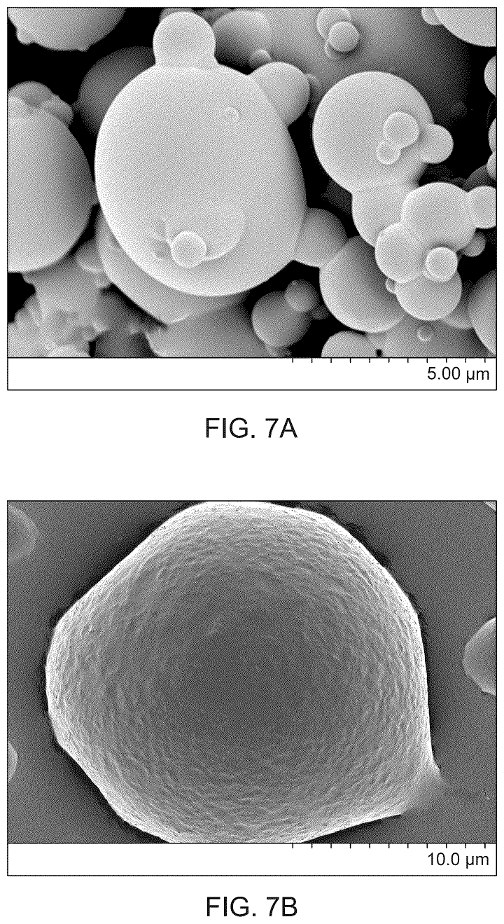

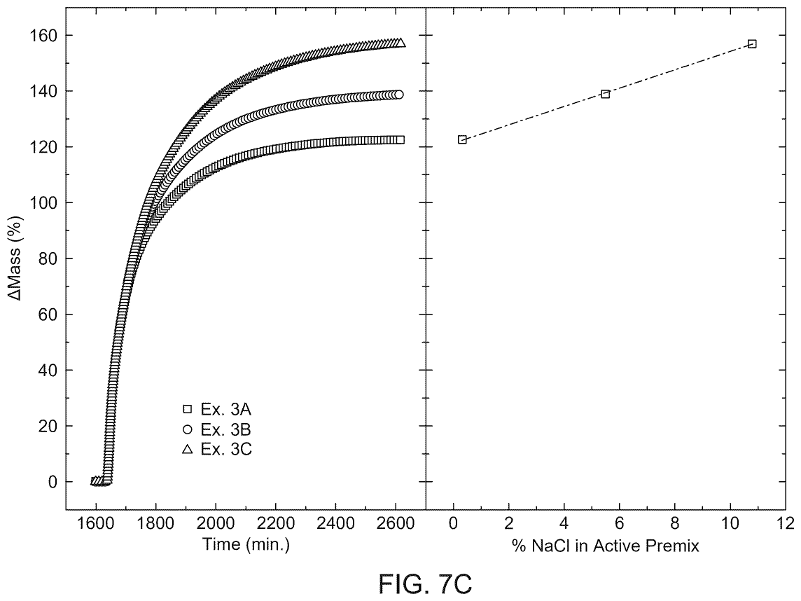

TABLE 11

| | Ex. | Ex. | Ex. | Ex. | Ex. | Ex. |
|---|---|---|---|---|---|---|
| | 3A | 3B | 3C | 4 | 5 | 6 |
| | APM | APM | APM | APM | APM | APM |
| Basic | ADCH | ADCH | ADCH | ASCH | ASCH | ASCH |
| Aluminum | | | | | | |
| Chloride Salt | | | | | | |
| % Al | 19.7 | 18.7 | 17.7 | 18.2 | 18.4 | 17.8 |
| % Cl (Total) | 26.18 | 28.11 | 30.05 | 23.29 | 23.14 | 22.43 |
| % Ca | 2.94 | 2.79 | 2.6 | 4.11 | 4.13 | 3.99 |
| % Glycine | 3.38 | 2.68 | 2.63 | | | |
| % Alanine | | | | 12.21 | | |
| % Beta- | | | | | 10.31 | |
| Alanine | | | | | | |
| % Trimethyl- | | | | | | 11.45 |
| glycine | | | | | | |
| Target % | 3.60 | 3.52 | 3.43 | 11.82 | 11.82 | 15.59 |
| Amino Acid | | | | | | |
| Al:Cl Atomic | 0.99 | 0.87 | 0.77 | 1.03 | 1.04 | 1.04 |
| Ratio | | | | | | |
| Al:Cl Atomic | 1.25 | 1.24 | 1.23 | | | |
| Ratio | | | | | | |
| of Active | | | | | | |
| % Na | 0.128 | 2.16 | 4.24 | 1.92 | 1.93 | 1.86 |
| Target % NaCl | 0 | 5 | 10 | 5 | 5 | 5 |
| Calculated % | 0.33 | 5.49 | 10.78 | 4.88 | 4.91 | 4.73 |
| NaCl | | | | | | |
| Ca:Al Atomic | 0.100 | 0.100 | 0.099 | 0.152 | 0.151 | 0.151 |
| Ratio | | | | | | |
| Amino Acid:Ca | 0.61 | 0.51 | 0.54 | 1.59 | 1.33 | 1.53 |
| Mol Ratio | | | | | | |
| Active pH | 3.93 | 4.03 | 4.00 | 4.68 | 4.62 | 4.66 |
| Peak 4:3 | 0.519 | 0.536 | 0.552 | 3.455 | 2.021 | 2.36 |
| Ratio | | | | | | |
| SEM Image | FIG. | FIG. | | FIG. | FIG. | FIG. |
| | 7A | 7B | | 8 | 9 | 10 |

| | |
|---|---|
| Kinetic Study | FIG. 7C |

Table 12 presents the maximum moisture absorption over a 48-hour period for the tested samples depicted in FIGS. 5C, 6C, and 7C, following the Water Vapor Sorption Test Method outlined in this document. The results indicate that the active premix samples containing a 10% sodium chloride concentration (e.g., Examples 1C, 2C, and 3C) exhibited superior moisture absorption compared to those with a 5% sodium chloride concentration (e.g., Examples 1B, 2B, and 3B) or no sodium chloride (e.g., Examples 1A, 2A, and 3A).

However, visual detection revealed that Examples 2C (the ASCH example with 10% NaCl) and Examples 3A-C (all ADCH examples) were excessively hygroscopic and had deliquesced, posing challenges for formulation in antiperspirant products. Furthermore, highly hygroscopic actives can lead to issues in aerosol antiperspirant formulations by potentially clogging the exit orifice. Notably, Example 2B (the ASCH sample with 5% NaCl) also showed signs of deliquescence, suggesting that a moisture pickup of approximately 106% may serve the approximate cutoff for ASCH.

There is variability in the hygroscopicity of basic aluminum chloride salts prior to the addition of sodium chloride, indicating that it may be beneficial to explore the addition of alternative salts, such as magnesium chloride, to enhance the usability of ASCH and ADCH in formulations.

TABLE 12

| | Target Added | Maximum | |
|---|---|---|---|
| | Sodium | Moisture | |
| | Chloride | Pickup over | Did the APM |
| | Concentration | 48 Hours | example |
| | (%) | (%) | deliquesce? |
| Ex. 1A ACH APM | 0 | 47 | No |
| Ex. 1B ACH APM | 5 | 62 | No |
| Ex. 1C ACH APM | 10 | 78 | No |
| Ex. 2A ASCH APM | 0 | 82 | No |
| Ex. 2B ASCH APM | 5 | 106 | The example |
| | | | showed signs |
| | | | of deliquescence |
| | | | but is still |
| | | | considered |
| | | | processable and |
| | | | the AP product |
| | | | will likely be |
| | | | consumer |
| | | | acceptable. |
| Ex. 2C ASCH APM | 10 | 124 | Yes |
| Ex. 3A ADCH APM | 0 | 123 | Yes |
| Ex. 3B ADCH APM | 5 | 139 | Yes |
| Ex. 3C ADCH APM | 10 | 157 | Yes |

### Combinations

A. An aerosol antiperspirant composition, comprising:
   a. a liquid propellant;
   b. an antiperspirant concentrate comprising:
      i. a particulate active premix composition comprising:
         1. a basic aluminum chloride salt of formula Al₂(OH)_{3.778}Cl_{2.222} to Al₂(OH)_{5.048}Cl_{0.952};
         2. an amino acid;
         3. greater than 1% of an alkali metal salt and/or magnesium chloride; and wherein the composition comprises a plurality of particles;
      ii. a carrier; and
      iii. a fragrance.
B. The aerosol antiperspirant composition according to Paragraph A, wherein the composition is anhydrous.
C. The aerosol antiperspirant composition according to Paragraphs A-B, wherein the antiperspirant composition comprises less than or equal to 25%, preferably from about 10% to about 24.5%, more preferably from about 12.5% to about 20%, even more preferably from about 12.5% to about 17.5%, by weight of the antiperspirant composition of the basic aluminum chloride salt.
D. The aerosol antiperspirant composition according to Paragraphs A-C, wherein the contrate comprises from about 8% to about 35%, from about 10% to about 33%, from about 12% to about 30%, or from about 15% to about 24%, by weight of the composition, of the carrier.
E. The aerosol antiperspirant composition according to Paragraphs A-D, wherein the carrier comprises a volatile silicone.
F. The aerosol antiperspirant composition according to Paragraph E, wherein the volatile silicone has a viscosity of less than 10 centistokes at 25° C.
G. The aerosol antiperspirant composition according to Paragraphs E-F, wherein the carrier comprises a cyclomethicone, preferably cyclopentasiloxane.
H. The aerosol antiperspirant composition according to Paragraphs E-F, wherein the carrier comprises dimethicone 5 cst.
I. The aerosol antiperspirant composition according to Paragraphs A-H, wherein the antiperspirant concentrate further comprises an emollient.
J. The aerosol antiperspirant composition according to Paragraph I, wherein the antiperspirant concentrate comprises from about 2% to about 7.5%, preferably from about 2.25% to about 6.75%, more preferably from about 2.5% to about 6%, even more preferably about 2.75% to about 5.5%, and most preferably from about 3% to about 5%, by weight of the aerosol antiperspirant composition, of the emollient.
K. The aerosol antiperspirant composition according to Paragraphs I-J, wherein the emollient is chosen from C12-15 alkyl benzoate, butyl myristate, butyl stearate, cetyl octanoate, diethylhexylcyclohexane, dicaprylyl carbonate, dicaprylyl ether, hydrogenated castor oil, isobutyl stearate, isopropyl myristate, isostearyl isostearate, isostearyl lactate, isostearyl palmitate, mineral oil, myristyl myristate, neopentyl glycol diheptanoate, octododecyl benzoate, octyldodecanol, petrolatum, PPG-14 butyl ether, or mixtures thereof, preferably PPG-14 butyl ether, C12-15 alkyl benzoate, isopropyl myristate, hydrogenated castor oil, or mixtures thereof.
L. The aerosol antiperspirant composition according to Paragraphs A-G and I-J, wherein the emollient comprises dimethicone 5 cst or greater.
M. The aerosol antiperspirant composition according to Paragraphs A-L, wherein the antiperspirant concentrate further comprises from about 0.6% to about 4.4%, preferably from about 0.7% to about 4.0%, more preferably from about 0.8% to about 3.6%, even more preferably from about 1% to about 3%, or most preferably from about 1.5% to about 2.5%, by weight of the aerosol antiperspirant composition, of a fragrance.
N. The aerosol antiperspirant composition according to Paragraphs A-M, wherein the composition comprises from about 60% to about 90%, preferably from about 60% to about 80%, by weight of the aerosol antiperspirant composition, of the liquid propellant.
O. The aerosol antiperspirant composition according to Paragraphs A-N, wherein the liquid propellant is chosen from 1,1 difluoroethane, propane, n-butane, isobutane, or mixtures thereof; preferably propane, n-butane, isobutane, or mixtures thereof.
P. The aerosol antiperspirant composition according to Paragraphs A-O, wherein the particulate active premix composition comprises up to 25%, preferably up to 20%, more preferably up to 15%, and most preferably up to 10%, by weight of the particulate active premix composition, of the alkali metal salt.
Q. The aerosol antiperspirant composition according to Paragraphs A-P, wherein the alkali metal salt chosen from sodium chloride, potassium chloride, lithium chloride, or mixtures thereof.
R. The aerosol antiperspirant composition according to Paragraphs A-Q, wherein the alkali metal salt comprises sodium chloride.
S. The aerosol antiperspirant composition according to Paragraphs A-R, wherein the particulate active premix comprises magnesium chloride.
T. The aerosol antiperspirant composition according to Paragraphs A-S, wherein the basic aluminum chloride salt comprises aluminum chlorohydrate salt of formula Al₂(OH)_{5.05}Cl_{0.95} to Al₂(OH)_{4.95}Cl_{1.05}.
U. The aerosol antiperspirant composition according to Paragraphs A-S, wherein the basic aluminum chloride salt comprises aluminum sesquichlorohydrate salt having an aluminum to chloride atomic ratio of from 1.26 to 1.90.
V. The aerosol antiperspirant composition according to Paragraphs A-S, wherein the basic aluminum chloride salt comprises aluminum dichlorohydrate salt having an aluminum to chloride atomic ratio of from 0.90 to 1.25.
W. The aerosol antiperspirant composition according to Paragraphs A-V, wherein the particulate active premix composition comprises from about 1% to about 25%, preferably from about 2% to about 20%, or more preferably from about 3% to about 18%, by weight of the particulate active premix composition, of the amino acid.
X. The aerosol antiperspirant composition according to Paragraphs A-W, wherein the amino acid is chosen from alanine, arginine, asparagine, aspartic acid, beta-alanine, trimethylglycine, cysteine, glutamic acid, glutamine, glycine, histidine, isoleucine, leucine, lysine, methionine, phenylalanine, proline, serine, threonine, tryptophan, tyrosine, valine, or mixtures thereof.
Y. The aerosol antiperspirant composition according to Paragraphs A-X, wherein the amino acid is chosen from glycine, alanine, trimethylglycine, or mixtures thereof.
Z. The aerosol antiperspirant composition according to Paragraphs A-Y, wherein the amino acid is chosen from glycine, alanine, beta-alanine, trimethylglycine, or mixtures thereof.
AA. The aerosol antiperspirant composition according to Paragraphs A-Z, wherein the amino acid is glycine.
BB. The aerosol antiperspirant composition according to Paragraphs A-AA, wherein the particulate active premix composition further comprises an alkaline earth metal salt.
CC. The aerosol antiperspirant composition according to Paragraph BB, wherein the particulate active premix composition comprises from about 0.5% to about 10%, preferably from about 1% to about 7%, more preferably from about 1.5% to about 5%, and even more preferably from about 1.75% to about 4.5%, by weight of the particulate active premix composition, of the alkaline earth metal salt.
DD. The aerosol antiperspirant composition according to Paragraphs BB-CC, wherein the alkaline earth metal salt is chosen from calcium chloride, calcium nitrate, calcium sulfate, strontium chloride, strontium sulfate, strontium nitrate, or mixtures thereof.
EE. The aerosol antiperspirant composition according to Paragraphs BB-DD, wherein the alkaline earth metal salt is chosen from calcium chloride, strontium chloride, or mixtures thereof.
FF. The aerosol antiperspirant composition according to Paragraphs BB-EE, wherein the alkaline earth metal salt comprises calcium chloride.
GG. The aerosol antiperspirant composition according to Paragraphs AA-FF, wherein the amino acid to alkaline earth metal molar ratio is ≤7, preferably ≤6, and more preferably ≤5.
HH. The aerosol antiperspirant composition according to Paragraphs AA-GG, wherein the amino acid to alkaline earth metal molar ratio is >0, preferably ≥0.25, more preferably ≥0.5, even more preferably ≥1.0, and even more preferably ≥1.5.
II. The aerosol antiperspirant composition according to Paragraphs AA-HH, comprising an alkaline earth metal to aluminum atomic ratio≥0.03, preferably ≥0.05, more preferably ≥0.06, and even more preferably ≥0.09.
JJ. The aerosol antiperspirant composition according to Paragraphs AA-II, comprising an alkaline earth metal to aluminum atomic ratio≤1, preferably ≤0.75, more preferably ≤0.5, even more preferably ≤0.35, and most preferably ≤0.2.
KK. The aerosol antiperspirant composition according to Paragraphs A-JJ, wherein the concentrate has a pH of greater than 3.5 to about 5.2, preferably from about 3.7 to about 5, more preferably from about 3.8 to about 4.8, even more preferably from about 4 to about 5, and most preferably from about 4.25 to about 4.75 as determined by the pH Test Method.
LL. The aerosol antiperspirant composition according to Paragraphs A-KK, wherein the particulate active premix composition has a maximum change in mass over 48 hours of ≥47%, preferably ≥55%, more preferably ≥65%, even more preferably ≥70%, and most preferably ≥75% according to the Water Vapor Sorption Test Method, described herein.
MM. The aerosol antiperspirant composition according to Paragraphs A-LL, wherein the particulate active premix composition has a maximum change in mass over 48 hours of ≤120%, preferably ≤115%, more preferably ≤110%, and even more preferably ≤106%, according to the Water Vapor Sorption Test Method, described herein.
NN. The aerosol antiperspirant composition according to Paragraph S, wherein each particle comprises aluminum, chloride, and sodium.
OO. The aerosol antiperspirant composition according to Paragraph NN, wherein each particle further comprises a surface coating and at least a portion of the sodium is present in the surface coating, preferably >50% of the sodium is present in the surface coating, more preferably >75% of the sodium is present in the surface coating.
PP. The aerosol antiperspirant composition according to Paragraphs NN-PP, wherein at least a portion of the sodium is interdispersed with the aluminum and chloride.
QQ. The aerosol antiperspirant composition according to Paragraph NN, wherein the particle is homogeneous.
RR. The aerosol antiperspirant composition of according to Paragraphs A-QQ, wherein the particulate active premix composition has a peak IV/III ratio of exceeding 0.75, preferably greater than or equal to 1, preferably greater than or equal to 1.5, preferably greater than or equal to 2, preferably greater than or equal to 2.5, preferably greater than or equal to 3, preferably greater than or equal to 3.25, preferably greater than or equal to 3.5, preferably greater than or equal to 3.75, and preferably greater than or equal to 3.85, according to the GPC Test Method.
SS. The aerosol antiperspirant composition according to Paragraphs A-RR, wherein the particulate active premix composition has a peak IV/III ratio of from about 1.5 to about 7.9, preferably from about 1.75 to about 5, more preferably from about 2 to about 4.5, more preferably from about 2.1 to about 4.25, more preferably from about 2.15 to about 4.1, even more preferably from about 2.25 to about 4, and even more preferably from about 2.4 to about 4.
TT. The aerosol antiperspirant composition according to Paragraphs A-EE, wherein the particulate active premix composition has a peak IV/III ratio of from about 2 to about 4, preferably from about 2.1 to about 3.75, more preferably from about 2.2 to about 3.5.
UU. An antiperspirant product comprising:
   a. a product container comprising
      i. a reservoir;
      ii. an actuator comprising a discharge orifice;
      iii. a valve in fluid communication with the discharge orifice and the reservoir, the reservoir storing the antiperspirant composition according to Paragraphs A-TT.
VV. The antiperspirant product according to Paragraph UU, wherein the product container has a pressure from about 60 kPa to about 500 kPA, preferably 200 kPa to about 400 kPa, more preferably 200 kPa to about 350 kPa at 25° C.
WW. The antiperspirant product according to Paragraph UU-VV, wherein the product container has a pressure from about 100 kPa to about 1100 kPA, preferably 200 kPa to about 1,100 kPa, or more preferably 400 kPa to about 900 kPa at 55° C.
XX. A non-therapeutic method of achieving a reduction in perspiration from the human body comprising the topical application of the aerosol antiperspirant composition according to Paragraphs A-TT.
YY. A method of attaining an antiperspirant benefit comprising the topical application to the surface of the human body, preferably to the axilla, of the antiperspirant composition according to Paragraphs A-TT.
ZZ. A method of topical application of an antiperspirant composition comprising the use the antiperspirant product according to Paragraphs UU-WW.
AAA. Use of the composition according to Paragraphs A-TT for antiperspirancy benefit.
BBB. A method of reducing corrosive damage to metal components of an aerosol product container comprising:
   a. providing the aerosol product container comprising
      i. a reservoir;
      ii. an actuator comprising a discharge orifice;
      iii. a valve in fluid communication with the discharge orifice and the reservoir, the reservoir storing the antiperspirant composition according to Paragraphs A-TT.

### Definitions

The term “anhydrous” as used herein in connection with an antiperspirant concentrate and/or antiperspirant composition refers to an antiperspirant concentrate that is substantially or completely free of added water, meaning water added as a separate ingredient to the antiperspirant concentrate. An anhydrous antiperspirant concentrate and/or composition may contain up to 10%, 8%, 6%, 4%, 2%, 15, or 0.5% water by weight of the antiperspirant concentrate that is bound with an ingredient (e.g., antiperspirant active, tapioca starch, etc.) added to the antiperspirant concentrate.

The term “aerosol antiperspirant product” refers to the combination of an aerosol antiperspirant product container, an antiperspirant neuter, a fragrance, and a liquid propellant stored in the aerosol antiperspirant product container.

Herein, “effective” means an amount of a subject active high enough to provide a significant positive modification of the condition to be treated. An effective amount of the subject active will vary with the particular condition being treated, the severity of the condition, the duration of the treatment, the nature of concurrent treatment, and like factors.

As used herein, the expressions “ranging from” and “between” are inclusive of the endpoints of the recited range(s).

As used herein, “visual detection” means that a human viewer can visually discern if the had deliquesced with the unaided eye (except for standard corrective lenses adapted to compensate for near-sightedness, farsightedness, or astigmatism, or other corrected vision) in lighting at least equal to the illumination of a standard 100-watt incandescent white light bulb at 30 cm.

All numbers expressing pH values are to be understood as being modified by the term “about,” and as encompassing readings using a pH meter having a variation of up to +10%, such as up to +9%, up to +8%, up to +7%, up to +6%, up to +5%, up to +4%, up to +3%, up to +2%, or up to +1%, which a skilled person will recognize relates to the inherent variation in pH meters.

All percentages are by weight of the composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 21° C. and at ambient conditions, where “ambient conditions” means conditions under about 1 atmosphere of pressure and at about 50% relative humidity. All weights as they pertain to listed ingredients are based on the active level and do not include carriers or by-products that may be included in commercially available materials, unless otherwise specified.

As used in this specification and the claims that follow, the articles “a”, “an”, and “the” include singular and plural references unless the context clearly dictates otherwise. As such, the terms “a” or “an”, “one or more” and “at least one” can be used interchangeably herein. Thus, for example, “a component” may include one or more components unless the reference is specifically indicated as being singular.

When a range of values is described herein, the range is contemplated to encompass the boundaries of the range as well as each value and sub-range within that range and individual values within that range. For example, “2 mg/L to 6 mg/L” is contemplated to encompass, for example, 2.0 mg/L, 2.5 mg/L, 2.7 mg/L, 3 mg/L, 4 mg/L, 4.5 mg/L, 5 mg/L, 5.5 mg/L, 6 mg/L, 2 mg/L to 3.5 mg/L, 2.5 mg/L to 4 mg/L, 2.5 mg/L to 4.5 mg/L, 4.5 mg/L to 6 mg/L, 4.8 mg/L to 5.3 mg/L, and so forth.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An aerosol antiperspirant composition, comprising:
a. A liquid propellant;
b. an antiperspirant concentrate comprising:
i) a particulate active premix composition comprising:
(1) a basic aluminum chloride salt of formula Al₂(OH)_{3.778}Cl_{2.222} to Al₂(OH)_{5.048}Cl_{0.952};
(2) an amino acid;
(3) an alkaline earth metal salt;
(4) greater than 1% of an alkali metal salt and/or magnesium chloride; and wherein the particulate active premix composition comprises a plurality of particles;
ii) a carrier; and
iii) a fragrance.

2. The aerosol antiperspirant composition of claim 1, wherein the composition is anhydrous.

3. The aerosol antiperspirant composition of claim 1, wherein the carrier comprises a volatile silicone.

4. The aerosol antiperspirant composition of claim 1, wherein the antiperspirant concentrate further comprises an emollient chosen from PPG-14 butyl ether, C12-15 alkyl benzoate, isopropyl myristate, hydrogenated castor oil, or mixtures thereof.

5. The aerosol antiperspirant composition of claim 1, wherein the liquid propellant is chosen from 1,1 difluoroethane, propane, n-butane, isobutane, or mixtures thereof.

6. The aerosol antiperspirant composition of claim 1, wherein the active premix composition comprises up to 25% of the alkali metal salt.

7. The aerosol antiperspirant composition of claim 6, wherein the active premix composition comprises the alkali metal salt chosen from sodium chloride, potassium chloride, lithium chloride, or mixtures thereof.

8. The aerosol antiperspirant composition of claim 7, wherein the alkali metal salt comprises sodium chloride and wherein each particle comprises aluminum, sodium, and chlorine.

9. The aerosol antiperspirant composition of claim 8, wherein each particle further comprises a surface coating and at least a portion of the sodium is present in the surface coating.

10. The aerosol antiperspirant composition of claim 1, wherein the composition comprises from about 5% to about 25% of the amino acid.

11. The composition of claim 1, wherein the amino acid is chosen from alanine, arginine, asparagine, aspartic acid, beta-alanine, trimethylglycine, cysteine, glutamic acid, glutamine, glycine, histidine, isoleucine, leucine, lysine, methionine, phenylalanine, proline, serine, threonine, tryptophan, tyrosine, valine, or mixtures thereof.

12. The composition of claim 1, wherein the alkaline earth metal salt is chosen from calcium chloride, calcium nitrate, calcium sulfate, strontium chloride, strontium sulfate, strontium nitrate, or mixtures thereof.

13. The composition of claim 1, wherein the basic aluminum chloride salt comprises aluminum chlorohydrate salt of formula Al₂(OH)_{5.05}Cl_{0.95} to Al₂(OH)_{4.95}Cl_{1.05}.

14. The composition of claim 1, wherein the basic aluminum chloride salt comprises aluminum sesquichlorohydrate salt having an aluminum to chloride atomic ratio of from 1.26 to 1.90.

15. The composition of claim 1, wherein the basic aluminum chloride salt comprises aluminum dichlorohydrate salt having an aluminum to chloride atomic ratio of from 0.90 to 1.25.

16. The composition of claim 1, wherein the has a peak IV/III ratio of from about 0.75 to about 7.9.

17. An antiperspirant product comprising:
a) a product container comprising
i) a reservoir;
ii) an actuator comprising a discharge orifice;
iii) a valve in fluid communication with the discharge orifice and the reservoir;
b) the antiperspirant composition of claim 1; wherein the reservoir stores the antiperspirant composition.

18. The antiperspirant product of claim 17, wherein the product container has a pressure from about 60 kPa to about 500 kPA at 25° C.

19. A method of providing sweat and odor protection comprising:
a. providing the antiperspirant product of claim 17;
b. spraying the antiperspirant concentrate onto a human's axilla.
